(19) United States
(12) Patent Application Publication
(10) Pub. No.: US 2004/0146523 A1
(43) Pub. Date: Jul. 29, 2004

(54) Use of the protein uk114 for the treatment and the prevention of chronic active hepatitis

(76) Inventors: Alberto Bartorelli, Crans sur Sierre (CH); Pierferdinando Nicoletti, Milano (IT); Alberto Panerai, Milano (IT)

Correspondence Address: YOUNG & THOMPSON, 745 SOUTH 23RD STREET 2ND FLOOR, ARLINGTON, VA, 22202

(21) Appl. No.: 10/474,364

(22) PCT Filed: Apr. 9, 2002
(86) PCT No.: PCT/EP02/03934

(30) Foreign Application Priority Data
Apr. 10, 2001 (IT) MI2001A 000761

(51) Int. Cl. A61K 39/29

(52) U.S. Cl. 424/189.1

(57) ABSTRACT

The use of the protein UK114, also known as MSP 14, for the preparation of medicaments for the treatment or the prophylaxis of chronic active hepatitis.

The present invention relates to the use of the protein UK 114, also known as MSP 14, for the preparation of medicaments for the treatment of chronic active hepatitis.

Chronic active hepatitis is an inflammatory process of the liver, particularly of portal spaces with parenchymal impairment and necrosis of the portal area, which is clinically characterized by evolutive poussées, with marked astenia, sub-jaundice and loss of weight, has protracted progression and results in portal fibrosis or cirrhosis. Two different forms of this disorder are known, which are classified as chronic hepatitis with moderate or intense evolution, respectively, depending whether the activity of the process is more or less marked. Both the intensity of inflammation and of the necrosis observed by hepatobiopsy and clinical and humoral signs are indications of the degree of activity. This disease occurs in both sexes, prevailing in the fourth/sixth decades of life.

The etiology of chronic active hepatitis is still unknown. However, a number of elements favor the presence of one or more liver-injuring immune factors. Chronic hepatitis can follow typical acute hepatitis (for example viral hepatitis) which may per se show no clinical manifestations and which may be followed by a short period of comparative well being (post-hepatitis or post-viral chronic hepatitis or secondary form). It is estimated that about 2-4% of acute hepatitis may evolve toward the chronic active form. On the other hand, in some cases no starting episodes are evidenced (chronic primary hepatitis or primary form). The factors favoring the development from the acute to the chronic form are still obscure, although (auto)immune factors play apparently a central role. To date, no therapeutical tools for the prevention and the therapy of the chronic active hepatitis are available, besides the protracted treatment with corticosteroids or cyclosporin A (which involve a number of side effects and can induce immune depression thus favoring the replication of the hepatic virus, if present). In consideration of the comparatively high percentage of acute hepatitis cases which evolve to chronic hepatitis, it would be particularly important to find an immune suppressive/immune modulating agent with low toxicity to be administered after recovery from acute hepatitis, to prevent a silent auto-reactive process against liver which could induce chronic active hepatitis in time.

A disease with clinical immunological and histological features similar to chronic active hepatitis can be experimentally induced in mice by a single intravenous injection of Concanavalin A (ConA) at the dose of 20 mg/kg. Between 8 and 24 hours after ConA injection, a massive lymphomonocytary infiltration of the liver is observed, which may be clinically evidenced by the marked increase in transaminase blood levels. ConA-induced hepatitis reaction is cell-mediated, in that this disease cannot be induced in athymic mice (lacking T lymphocytes) and may be prevented by cyclosporin A or silica which block macrophages activity.

It has now been found that the protein of molecular weight 14 kDa in SDS-PAGE, obtainable by extraction of mammalian liver with perchloric acid, named UK114, disclosed in WO 96/02567 and in WO 00/63368, which has to date been studied for a series of therapeutical applications including treatment of neoplasias (WO 96/02567), autoimmune diseases (WO 98/11909), TNF-induced pathologies (WO 98/42366), AIDS (WO 98/11137), treatment and prevention of the rejection of transplanted organs (WO 00/78329), can be advantageously used in the treatment of chronic active hepatitis.

Therefore, the invention relates to pharmaceutical compositions for the treatment of the chronic active hepatitis, containing as active ingredient the protein UK 114 of extractive origin, alone or in combination with other components, or the recombinant protein UK 114, disclosed in WO 00/63368.

For the envisaged therapeutical use, the protein UK 114 will preferably be administered parenterally, at doses which will be easily determined by the physicians based on the pharmacokinetic and toxicological characteristics of the protein, as well as by the severity of the disease and the conditions of the patient (weight and age). In principle, dosages will range between 0.1 and 10 mg a day of UK 114, intramuscularly or subcutaneously, optionally divided into more administrations.

The pharmacological activity of UK 114 was evidenced in the Concanavalin A-induced hepatitis model as reported in the following example.

### EXAMPLE

Six to eight weeks male Balb/c mice were injected intraperitoneally with 30 micrograms of UK114 (in 100 μL of PBSI) 24 hours and 1 hour before intravenous administration of 20 mg/kg ConA (group A). A group of control mice was treated with 100 μl of PBS according to the same experimental procedures used for UK114 (group B). A further group of mice was injected with PBS instead of ConA (group C). Mice were killed 8 hours after ConA administration and blood samples were collected from the single animals for evaluating alanine aminotransferase (ALAT) levels. At the same time, liver was taken for histological examination in hematoxylin/eosin.

As shown in the table below, intravenous administration of ConA, contrary to PBS, induced a marked increase in ALAT plasma levels. Histological examination of the liver showed severe lymphomonocytary infiltration. The prophylactic treatment with UK 114 drastically prevented any increase in serum transaminases. In agreement to these data, histological examination revealed a marked reduction in the number of lymphomonocytes infiltrating liver in the animals treated with UK114.

These data prove that the administration of UK114 is capable of preventing the development of ConA-induced hepatitis in Balb/c mice, which is, as already mentioned, a known, established animal model of chronic active hepatitis. Therefore UK114 may be advantageously used in the first phases of chronic active hepatitis, and likely also in the prophylaxis of the phases following viral hepatitis which, as mentioned above, can frequently evolve to chronic active hepatitis.

TABLE
Effects of the prophylactic treatment with UK114 on
the development of ConA-induced autoimmune hepatitis

| Group (n) | ConA (20 mg/Kg) | Treatment | ALAT* (U/I) |
|---|---|---|---|
| A (20) | + | UK114 | 560 ± 147** |
| B (40) | + | PBS | 4012 ± 392 |
| C (30) | − | Nil | 57 ± 10 |

*alanine aminotransferase, iSD mean values
**p < 0.0001 with Student t test vs group B

### LITERATURE

1) Ferluga J. et al. Lancet, 1978, 2:610;

2) Tiegs G. et al. J. Clin. Invest. 1992, 90:196;

3) Mizuahara H. et al. J. Exp. Med., 1994, 179:1529;

4) Panerai A. E. et al. Ann. NY Acad. Sci. 1999, 876:229.

1. The use of the protein UK 114 for the preparation of medicaments for the treatment or the prophylaxis of chronic active hepatitis.

2. The use as claimed in claim 1 wherein the protein is of extractive or recombinant origin, and is optionally combined with other components.

3. The use as claimed in claim 1 or 2 for the preparation of medicaments for the prophylactic treatment in the phases following viral hepatitis.
